SEQUENCE LISTING (16 sequences)

SEQ ID NO: 1 - protein, 814 aa, Comamonas sp.E6
METALAARGILETHRALASERVALPHETHRSERHISVALPRALAILEGLYALAALAMETASPMETGLYLYSTHRGLNGLALATYRTRPALAPRLEPHELYSGLYTYRASPPHESERARGGLNTRPMETLYSASPASNLYSPRASPVALILEPHELEVALTYRASNASPHISALATHRALAPHESERLEASPCYSILEPRTHRPHEALAILEGLYTHRALAALAGLPHEGLNPRALAASPGLGLYTRPGLYPRARGPRVALPRLYSVALVALGLYHISPRASPLEALASERHISILEALAGLNSERVALILEGLNGLNASPPHEASPLETHRILEVALASNLYSMETASPVALASPHISGLYLETHRVALPRLESERLEMETCYSGLYGLGLNASPPRLYSTHRGLYSERTRPPRCYSPRVALILEPRPHEALAVALASNVALVALGLNTYRPRVALPRTHRGLYGLNARGCYSPHEASNLEGLYARGALAILEARGLYSALAVALGLSERTYRASPGLNASPILEASNVALHISILETRPGLYTHRGLYGLYMETSERHISGLNLEGLNGLYALAARGALAGLYLEILEASNLYSGLTRPASPASNGLNPHELEASPLELEILEGLASNPRHISGLYLEALAGLNMETPRHISILEASPTYRVALARGGLALAGLYSERGLGLYILEGLLEVALMETTRPLEILEALAARGGLYALAMETSERASPVALASPGLYPRALAPRLEPRLYSVALALAHISARGPHETYRHISVALPRALASERASNTHRALAVALGLYHISLEILELEGLASNGLN

SEQ ID NO: 2 - DNA, 870 bp, Comamonas sp.E6
atggcacgcatcaccgcatccgttttcacctcgcacgtgcctgccatcggcgccgccatggacatgggcaagacccaggaagcctactgggcgcccctgttcaagggttatgacttctcccgccagtggatgaaggacaacaagcccgatgtgatcttcctggtctacaacgaccacgccacggccttcagcctggactgcattcccaccttcgccatcggcacggctgcggaattccagcccgccgacgaaggctggggcccgcgccccgtgcccaaggtggtcggccatcccgatctggccagccacattgcccagtccgtgatccagcaggacttcgatctgaccatcgtcaacaagatggacgtggaccacggcctcacggtgcctctgtcgctgatgtgcggcgagcaggaccccaagaccggctcctggccctgcccggtgatccccttcgccgtgaacgtggtgcagtatcccgtgcccaccggccagcgctgcttcaacctgggccgcgccatccgcaaggccgtggagagctacgaccaggacatcaacgtccatatctggggcacgggtggcatgagccaccagctgcagggtgcgcgcgcaggcctgatcaacaaggaatgggacaaccagttcctggacctgctgatcgagaacccccacggtctggcgcagatgccgcacatcgactacgtgcgcgaagccggctcggaaggcatcgagctggtgatgtggctgattgcgcgcggcgccatgtccgatgtggacggccccgcaccgctgcccaaggtggcgcaccgcttctaccatgtgcctgcatcgaacaccgcggtgggccatctgatcctcgagaatcagtaa

SEQ ID NO: 3 - protein, 425 aa, Comamonas sp.E6
METALALEGLLYSPRTYRLEASPVALPRGLYTHRILEILEPHEASPALAGLGLNSERARGLYSGLYTYRTRPLEASNGLNPHECYSMETSERLEMETLYSALAGLASNARGGLARGPHEARGALAASNGLARGALATYRLESERGLTRPALAMETARGGLGLGLNLYSGLNALAVALLEALAARGASPLEASNTRPCYSMETARGTHRGLYGLYASNILETYRPHELEALALYSILEGLYALATHRASPGLYLYSSERPHEGLNGLNMETALAGLYSERMETTHRGLYMETTHRGLGLGLTYRARGALAMETMETMETGLYGLYGLYARGSERALAASPGLYASNARGTYRVALGLYGLASPGLYASPALAGLNALAILEILEARGGLNPRGLNGLYSERALAGLYASNGLNASNGLGLGLYASN

SEQ ID NO: 4 - DNA, 450 bp, Comamonas sp.E6
atggctttggaaaaaccgtatctggacgtgcccggcaccatcattttcgatgccgagcagtcccgcaagggctactggctcaaccagttctgcatgagcctgatgaaggccgagaaccgcgagcgctttcgcgccaacgagcgtgcctatctggacgagtgggcgatgaccgaggagcagaagcaggccgtgctggcgcgtgacctgaactggtgcatgcgcaccggcggcaatatctacttcctggccaagattggcgccaccgacggcaagagcttccagcagatggcgggctccatgaccggcatgaccgaagaagagtaccgcgccatgatgatgggcggcggccgctctgccgatggcaatcgctatgtgggcgaggacggtgatgcgcaggcgcatcgccagccccagggcagcgcaggcaaccagaacaaggaaggcaactaa

SEQ ID NO: 5 - DNA, 26 bp, Artificial Sequence
  aF Primer
atgwssctgatgaarscsgaraaccg

SEQ ID NO: 6 - DNA, 27 bp, Artificial Sequence
  bF Primer
gtswtcctggtstayaacgaycaygcy

SEQ ID NO: 7 - DNA, 25 bp, Artificial Sequence
  bR Primer
ccctgmarctgrtggctcatgccgc

SEQ ID NO: 8 - DNA, 5386 bp, Comamonas sp. E6
atgatcattgacgtacacggtcactacaccacggcgcctgcggctctgggcgcatggcgcgatctgcagatcgccggcctcaaggacccgagcaagaccccgtcggtggccgatctgaagatcagcgacgatgaaatccgcgagaccatcgaaaccaatcagctgcgcctgatgaaggagcgtggttccgatctgaccatcttcagcccccgtgcctcgttcatggcgcaccacatcggtgacttccagacctccagcacctgggccgccatctgcaacgagctgtgcttccgcgtcagcgagctgttccccgaccacttcattcccgccgccatgctgccccagtcgcccggcgtggaccctgcaacctgcattcccgagctggtcaagtgcgttgagcaatatggcaacgtgggcctgaacctgaaccccgatccctcgggcggtcactggacttcgcctccgctgtccgacaagagctggtaccccatctacgaaaagatggtggagtacgacatccccgcgatgatccacgtctccaccagctgcaatagctgcttccacaccacgggcagccactatctgaatgccgacaccacggccttcatgcagtgcctgacttcggatctgttcaaggacttcccgaccctgaagttcctgattccccatggcggcggcgccgtgccttaccactggggccgtttccgcggtctggcgcaggagatgaagaagccgctgctggaagagcatttgctcaacaacatctacttcgacacctgcgtctaccaccagccgggcatcaacctgctgacggaagtgattccgaccaagaacattctgttcgccagcgaaatgatcggcgccgtgcgcggcatcgatccgcagaccggtcactactacgacgacaccaagcgctacatcgaagccacgcagaacctgacggccgatgaaaagcacgccgtctacgaaggcaatgcccgccgcgtgttcacgcgcctggacaaggcgctcaaggccaagggtctgtaacttctaaaaaagagagctgctggcgcacggtatgaaaggttttcaataagaaaactattcagatcttatgaatagcaagcgctaacagctcactttttagattcaataacgtcaaagcaaagaggtatttccatgtacgaactgggagttgtctaccgcaatatccagcgcgccgaccgcgctgctgctgacggcctggccgccctgggctctgccaccgtgcacgaggccatgggccgcgttggtctgctcaagccctatatgcgccccatctatgccggcaagcaggtctcgggtacggccgtcacggtgctgctgcagcccggcgacaactggatgatgcatgtggctgccgagcagattcagcccggcgatatcgtggtcgctgccgtcaccgccgagtgctccgacggctacttcggcgacctgctggccaccagcttccaggcgcgcggcgcacgcgcgctgatcatcgatgccggcgtgcgcgacgtgaagacgctgcaggagatggacttcccggtctggagcaaggccatctcttccaagggcacgatcaaggccaccctgggctcggtcaacatccccatcgtctgcgccggcatgctggtcacgcccggtgacgtgatcgtggccgacgacgacggcgtggtctgcgtgcccgccgcgcgtgccgtggaagtgctggccgctgcccagaagcgcgaaagcttcgaaggcgaaaagcgcgccaagctggcttcgggcgtcctgggcctggatatgtacaagatgcgcgagcccctggaaaaggccggcttgcgctatgtggattaactccccctgagcggctgcgccgcttccccctctctcgctgcgcgggaaggggacgacaccctcggtgcggggcggcccttcctcggtgtctctgagatgtggcagcgccagtttcatggatggtgggaagtgcgcagcgcacggtgcaatcaacaattgaggagcaaacagatgagcgcctttgaaaaaacccccggctggctggactggtatgccaaccccagcaagccccagttcaagctgcctgccggcgctgtggatgcgcactgccatgtgttcggtcccggtaacgagttccccttcgcccccgagcgcaagtacaccccctgcgacgccagcaaggcccagctgtatgcgctgcgcgaccatctgggttttgcgcgcaatgtggtggtgcaggccacctgccacggtgcggacaaccgcgccatggtcgatgcctgcaagtcctcgggcggcaaggcccgcggcgtggccacggtcaagcgctccatcagcgatgccgaactgcaggagctgcatgacgccggcgtgcgcggcgtgcgtttcaacttcgtcaagcgtctggtggactttacgcccaaggacgagctgatggagatcgccggtcgcatcgccaagctgggctggcatgtggtgatctatttcgaagccgtggatctgcccgagctgtgggacttcttcaccgcgctgcccaccaccgtggtggtcgaccacatgggccgccccgacgtgaccaagggcgtggacagcgaggagttcgccctgttcttgaagttcatgcgcgagcacaagaatgtctggagcaaggtttcctgccccgagcgcctgtccgtctccggccccaaggcgcttcatggtgagcagaacgcctaccaggacgtggtgcctttcgcgcgccgcgtggtcgaggagttccccgagcgcgtgctctggggcacggactggccgcaccccaacctgaaggaccacatgcccgacgacggcctgctggtggacttcattcctcatatcgctcctaccgcgcagctgcagcaaaagctgctggtggacaaccccatgcgtctgtactggcccgaagaggtctgacggagttccggcgacacatacacccggaaacaggccgcagccttgtcgcatgggctgcgccatccgtaacgaggagaatttatggctttggaaaaaccgtatctggacgtgcccggcaccatcattttcgatgccgagcagtcccgcaagggctactggctcaaccagttctgcatgagcctgatgaaggccgagaaccgcgagcgctttcgcgccaacgagcgtgcctatctggacgagtgggcgatgaccgaggagcagaagcaggccgtgctggcgcgtgacctgaactggtgcatgcgcaccggcggcaatatctacttcctggccaagattggcgccaccgacggcaagagcttccagcagatggcgggctccatgaccggcatgaccgaagaagagtaccgcgccatgatgatgggcggcggccgctctgccgatggcaatcgctatgtgggcgaggacggtgatgcgcaggcgcatcgccagccccagggcagcgcaggcaaccagaacaaggaaggcaactaagacatggcacgcatcaccgcatccgttttcacctcgcacgtgcctgccatcggcgccgccatggacatgggcaagacccaggaagcctactgggcgcccctgttcaagggttatgacttctcccgccagtggatgaaggacaacaagcccgatgtgatcttcctggtctacaacgaccacgccacggccttcagcctggactgcattcccaccttcgccatcggcacggctgcggaattccagcccgccgacgaaggctggggcccgcgccccgtgcccaaggtggtcggccatcccgatctggccagccacattgcccagtccgtgatccagcaggacttcgatctgaccatcgtcaacaagatggacgtggaccacggcctcacggtgcctctgtcgctgatgtgcggcgagcaggaccccaagaccggctcctggccctgcccggtgatccccttcgccgtgaacgtggtgcagtatcccgtgcccaccggccagcgctgcttcaacctgggccgcgccatccgcaaggccgtggagagctacgaccaggacatcaacgtccatatctggggcacgggtggcatgagccaccagctgcagggtgcgcgcgcaggcctgatcaacaaggaatgggacaaccagttcctggacctgctgatcgagaacccccacggtctggcgcagatgccgcacatcgactacgtgcgcgaagccggctcggaaggcatcgagctggtgatgtggctgattgcgcgcggcgccatgtccgatgtggacggccccgcaccgctgcccaaggtggcgcaccgcttctaccatgtgcctgcatcgaacaccgcggtgggccatctgatcctcgagaatcagtaaacgtttcaccacgttcgctgcttcgcgtattcactgccccctgtggggggcttcgcctccttgaggcggctctacggagatcccaatcatgagcaagaccatcaaagtagcgctggctggcgcaggtgccttcggcatcaagcacctggacggcatcaagaacatcgacggcgtggaagtcgtctccctggtcggtcgccgctttgaccagaccaaggaagtggccgacaaatacggcatcaagcatgtggcaaccgatctggccgaaagcctggcgctgcccgaggtcgatgccgtgatcctttgcacgcccacgcagatgcacgccgagcaggccattgcctgcatgaaggccggcaagcatgtgcaggtcgagattcctctggccgatgccctgaaggacgcccaggaagtggccgagctgcaaaagcagaccggtctggtggccatggtgggtcacacccgccgcttcaaccccagccaccagtgggtgcacaagaagatagcagccggcgagttcaacatccagcagatggatgtgcaaacctacttcttccgccgcaccaatatgaacgcgctgggccaggcccgcagctggaccgaccacctgctgtggcaccatgccgcccacaccgtggacctgttcgcctaccaggccggcagccccatcgtcaaggccaacgccgtgcaaggcccgattcacaaggatctgggcatcgccatggacatgagcatccagctcaaggccgccaacggcgcgatctgcacgctgagcctgtcgttcaacaatgacggccctctgggtaccttcttccgctacatcggcgacaccggcacctatctggcccgctacgacgatctgtacaccggcaaggacgagaagatcgacgtgtcccaggtcgatgtgtccatgaacggcatcgagctgcaggaccgcgaattcttcgccgccatccgtgaaggccgcgagcccaactccagcgtgcagcaggtgttcaactgctacaaggtcttgcacgacctggagcagcaactcaacgccgaataa

SEQ ID NO: 9 - DNA, 768 bp, Comamonas sp.
atgcaggacaagaactttgtggaatcgctgcgcaagggattgggggtactgacttgctttgaccgtcggcatacccggctgacgctgtcagaggtagccaggctcacgcagtccacgccagcatccgccagacgttcgctcagcacactggtacagcttggctatctagagagcgacggcaaactgttctggatgcagcccaaatcgctgctgatcgcctattcatttctgtcatcgcgccccatgcctgcattggcccagccactactggatgcactgtcggagcgcaccagagaatccgcttcgcttggtactttgttggaggacgatgccatcatcattggtcgttcgaccgcacggcgcagcttgagcacgggcctaggaataggatctaggttgccggtgtactgctctgcgattggtcggatgctgttgtcaggactcccccaacaggaggcgcgtgcaaggctagagatgatcgagcgggtggcactgacccctcatacggtgactgacttggaggagctgctaggtctgcttgaaacttgccggcaatcagggtggtcatgcagcgacggagagctggagctgggggtgcgctctatggcagcgccagtgcgcgaccctcaaggcaacacaattgctgccatgagcattgctgttagggcagagagactcagcatgagtgagttcaaggagacttttttgataccgctgaagcgcgctcgcaatgagttggaaaaaaagctatatccgcaggggttgtag

SEQ ID NO: 10 - DNA, 969 bp, Comamonas sp.
atgcgcaacgaatctattcgcagacgcgaagcattgatcggcattgccgctgcggtcgctgcaacaggtagcctggctcagtcaaatcaacctctcaagatcgtcgtgcctttctcagcgggaggtaccgccgatgtactgccgcgcctagtagccgaaaaaatccgcgcggactacgcaggtggcgtgatcattgaaaacaagcccggggctggtggaaacatcggtgccgatctagtgttccgggcaccgccagacggaatgactgtgctagcctcccctccggggcccatcgccatcaaccacaacctctatcaaaagctcagcttcgacccgactcgctgggtaccggtgacgattttggccacagtgcccaacgtcctggtcatcaacccgaagctgcctgttaagtcacttggcgagttcatcgcatacgccaaagccaatcccaaaaaagtgactgtggcgacgcaaggcgacggctccacatcccacctgactgctgccatgtttatgcagctgactggcactgaactgactgtcatcccttacaagggaacagccccggcactgattgacttgatcggcggcaatgtggatgtgtttttcgacaacatcagctcgtcggccacctaccaccaggccggcaaggtgcgcattctggccgttgccgacgagcagcgctctcaaatattgccccaggtccccaccttcgccgagcagcaatggcccgccatgcaagccgtgactttcttttctgttgtggctcccccaggcacgagtgcagaaatcgctcagaagcttcagaagcagatggccctggctctgtcatcgaacgacatccgcaagcactttcaggaacaaggtgccgtgccttgtggttgggacccgtccaagactgctcagttcatccgccaggaaaccgagaagtggaagaaggtgctgaaggccgccaacgtcaagctctaa

SEQ ID NO: 11 - DNA, 1242 bp, Comamonas sp.
atgcaagaatccatcatccagtggcatggggccactaatacgcgcgtgccttttggtatctataccgacacagccaatgctgatcaggaacagcagcgcatctatcgcggcgaggtctggaactacttgtgcctggaatctgaaattcccggggccggtgatttccgcactacctttgccggtgaaacaccgatagttgtcgtacgggatgccgaccaggaaatctacgccttcgagaaccgctgcgcgcatcgcggcgctctcatcgctctggagaaatcgggccgtacggatagtttccagtgcgtctatcacgcctggagctacaaccgacagggagatctgaccggcgttgccttcgagaaaggtgtcaagggccagggtggcatgccggcctcattctgcaaagaagagcatggcccgcgcaagctccgcgtggctgtcttttgcggtttggtctttggcagtttttccgaggacgtgcccagcattgaggattaccttggccctgagatttgcgagcgcatagagcgcgtgctgcacaagcccgtagaagtcatcggtcgcttcacgcaaaagctgcctaacaactggaagctctacttcgagaacgtgaaggacagctatcacgccagcctcctgcatatgttcttcaccaccttcgagctgaatcgcctctcacaaaaaggcggtgtcatcgtcgacgagtcgggtggccaccatgtgagctattccatgatcgatcgcggcgccaaagacgactcgtacaaggaccaggccatccgctccgacaacgagcgttaccggctcaaagatcctagccttctagagggctttgaggagttcgaggacggcgtgaccctgcagatcctttctgtgttccctggctttgtgctgcagcagattcagaacagcatcgccgtgcgtcagttgctgcccaagagcatctccagctcggaactcaactggacctatcttggctatgcagatgacagtgccgagcaacgcaaggtcagactcaaacaggccaaccttatcggcccggccggattcatttccatggaagacggagctgtcggtggattcgtgcagcgtggcatcgcaggcgctgccaaccttgatgcagtcatcgagatgggcggagaccacgaaggctctagcgagggccgcgccacggaaacctcggtacgcggcttttggaaggcctaccgcaagcatatgggacaggagatgcaagcatga

SEQ ID NO: 12 - DNA, 465 bp, Comamonas sp.
atgatcaatgaaattcaaatcgcggccttcaatgccgcctacgcgaagaccatagacagtgatgcaatggagcaatggccaacctttttcaccaaggattgccactattgcgtcaccaatgtcgacaaccatgatgagggacttgctgccggcattgtctgggcggattcgcaggacatgctcaccgaccgaatttctgcgctgcgcgaagccaatatctacgagcgccaccgctatcgccatatcctgggtctgccttcgatccagtcaggcgatgcaacacaggccagcgcttccactccgttcatggtgctgcgcatcatgcatacaggggaaacagaggtctttgccagcggtgagtacctcgacaaattcaccacgatcgatggcaagttacgtctgcaagaacgcatcgcggtttgcgacagcacggtgacggacacgctgatggcattgccgctatga

SEQ ID NO: 13 - DNA, 948 bp, Comamonas sp.
atgacaatagtgcaccgtagattggctttggccatcggcgatccccacggtattggcccagaaatcgcactgaaagctctccagcagctgtctgtcaccgaaaggtctcttatcaaggtctatggaccttggagcgctctcgagcaagccgcacgggtttgcgaaatggagccgcttcttcaagacatcgttcacgaggaagccggcacacttacacaaccagttcaatggggagaaatcaccccgcaggctggtctatctacggtgcaatccgcaacagcggctatccgagcgtgcgaaaacggcgaagtcgatgccgtcattgcctgccctcaccatgaaacggccattcaccgcgcaggcatagcgttcagcggctacccatctttgctcgccaatgttcttggcatgaacgaagaccaggtattcctgatgctggtgggggctggcctgcgcatagtgcatgtcactttgcatgaaagcgtgcgcagcgcattggagcggctctctcctcagttggtggtcaacgcggcgcaggctgccgtgcagacatgcaccttactcggagtgcctaaaccaaaagtcgctgtattcgggatcaaccctcatgcatctgaaggacagttgttcggcctggaggactcccagatcaccgttcccgctgtcgagacactgcgcaagcgcggcctagcagtagacggccccatgggagctgacatggttctggcacagcgcaagcacgacctgtatgtagccatgctgcacgatcagggccatatccccatcaagctgctggcacctaacggagccagcgcactatctatcggtggcagggtggtgctttccagcgtgggccatggcagcgccatggacattgccggccgtggcgtggctgacgccacggccctcctacgcacaatagccctactcggagcccaaccggtctga

SEQ ID NO: 14 - DNA, 1011 bp, Comamonas sp.
atgaaccaccagatccatatccacgactccgatatcgcgttcccctgcgcgcccgggcaatccgtactggatgcagctctgcaggccggcatcgagctgccctattcctgccgcaaaggtagctgtggcaactgtgcgagtacgctgctcgacggaaatattgcctccttcaatggcatggccgtgcgaaacgaactctgcgcctcggaacaagtgctgctgtgcggctgcactgcagccagcgatatacgtatccacccgagctcctttcgccgtctcgacccggaagcccgaaaacgttttacggccaaggtgtacagcaatacactggcggcacccgatgtctcgctgctgcgcctgcgcctgcctgtgggcaagcgcgccaaatttgaagccggccaatacctgctgattcacctcgacgacggggaaagccgcagctactctatggccaatccaccccatgagagcgatggcatcacattgcatgtcaggcatgtacctggtggtcgcttcagcactatcgttcagcagttgaagtctggtgacacattggatatcgaactgccattcggcagcatcgcactgaagcctgatgacgcaaggcccctgatttgcgttgcgggtggcacgggatttgcgcccattaaatccgttcttgatgacttagccaaacgcaaggttcagcgcgacatcacgctgatctggggggctcgcaacccctcgggcctgtatcttcctagcgccatcgacaagtggcgcaaagtctggccacagtttcgctacattgcagccatcaccgacctaggcgatatgcctgcggatgctcacgcaggtcgggtggatgacgcgctacgcactcactttggcaacctgcacgatcatgtggtgcactgctgtggctcaccagctctggttcaatcagtgcgcacagccgcttccgatatgggcctgcttgcacaggacttccacgcggatgtttttgcgacaggcccgactggtcaccactag

SEQ ID NO: 15 - DNA, 720 bp, Pseudomonas putida KT2440
atgcccgcccaggacaacagccgcttcgtgatccgtgatcgcaactggcaccctaaagcccttacgcctgactacaagacctccgttgcccgctcgccgcgccaggcactggtcagcattccgcagtcgatcagcgaaaccactggtccggacttttcccatctgggcttcggcgcccacgaccatgacctgctgctgaacttcaataacggtggcctgcccattggcgagcgcatcatcgtcgccggccgtgtcgtcgaccagtacggcaagcctgtgccgaacactttggtggagatgtggcaagccaacgccggcggccgctatcgccacaagaacgatcgctacctggcgcccctggacccgaacttcggtggtgttgggcggtgtctgaccgaccgtgacggctattacagcttccgcaccatcaagccgggcccgtacccatggcgcaacggcccgaacgactggcgcccggcgcatatccacttcgccatcagcggcccatcgatcgccaccaagctgatcacccagttgtacttcgaaggtgacccgctgatcccgatgtgcccgatcgtcaagtcgatcgccaacccgcaagccgtgcagcagttgatcgccaagctcgacatgagcaacgccaacccgatggactgcctggcctaccgctttgacatcgtgctgcgcggccagcgcaagacccacttcgaaaactgctga

SEQ ID NO: 16 - DNA, 606 bp, Pseudomonas putida KT2440
atgccaatcgaactgctgccggaaaccccttcgcagactgccggcccctacgtgcacatcggcctggccctggaagccgccggcaacccgacccgcgaccaggaaatctggaactgcctggccaagccagacgccccgggcgagcacattctgctgatcggccacgtatatgacggaaacggccacctggtgcgcgactcgttcctggaagtgtggcaggccgacgccaacggtgagtaccaggatgcctacaacctggaaaacgccttcaacagctttggccgcacggctaccaccttcgatgccggtgagtggacgctgcaaacggtcaagccgggtgtggtgaacaacgctgctggcgtgccgatggcgccgcacatcaacatcagcctgtttgcccgtggcatcaacatccacctgcacacgcgcctgtatttcgatgatgaggcccaggccaatgccaagtgcccggtgctcaacctgatcgagcagccgcagcggcgtgaaaccttgattgccaagcgttgcgaagtggatgggaagacggcgtaccgctttgatatccgcattcagggggaaggggagaccgtcttcttcgacttctga

(19) United States
(12) Patent Application Publication
(10) Pub. No.: US 2010/0209978 A1
(43) Pub. Date: Aug. 19, 2010

(54) GENE-DISRUPTED STRAIN, RECOMBINANT PLASMIDS, TRANSFORMANTS AND PROCESS FOR PRODUCTION OF 3-CARBOXYMUCONOLACTONE

(75) Inventors: Toshihisa Shimo, Kariya-shi (JP); Kohei Mase, Kariya-shi (JP); Yoshihiro Katayama, Tokyo (JP); Eiji Masai, Nagaoka-shi (JP); Masao Fukuda, Nagaoka-shi (JP); Kiyotaka Shigehara, Tokyo (JP); Seiji Ohara, Tsukuba-shi (JP); Masaya Nakamura, Tsukuba-shi (JP); Yuichiro Otsuka, Tsukuba-shi (JP)

Correspondence Address: Locke Lord Bissell & Liddell LLP;Attn: IP Docketing, Three World Financial Center, New York, NY, 10281-2101, US

(73) Assignees: KABUSHIKI KAISHA TOYOTA JIDOSHOKKI, Aichi-ken (JP); NAGAOKA UNIVERSITY OF TECHNOLOGY, Niigata (JP); FORESTRY AND FOREST PRODUCTS RESEARCH INSTITUTE, Ibaraki (JP)

(21) Appl. No.: 12/599,787

(22) PCT Filed: May 9, 2008
(86) PCT No.: PCT/JP2008/058988

§ 371 Date: Nov. 11, 2009

(30) Foreign Application Priority Data
May 11, 2007 (JP) 2007-126990

(51) Int. Cl. C12P 7/44; C12N 1/21; C12N 15/63; C12P 17/02

(52) U.S. Cl. 435/123; 435/252.3; 435/320.1; 435/142

(57) ABSTRACT

Industrial-scale fermentative production of 3-carboxy-cis,cis-muconic acid from terephthalic acid. Also, a protocatechuate 4,5-ring-cleaving enzyme gene-disrupted strain in which the gene coding for (a) the amino acid sequence set forth in SEQ ID NO: 1 or 3, or (b) the amino acid sequence set forth in SEQ ID NO: 1 or 3 which has a deletion, substitution, addition and/or insertion of one or more amino acids and exhibits protocatechuate 4,5-ring cleavage activity, present in the chromosomal DNA of microbial cells, has been disrupted; recombinant plasmids comprising the Tph gene and protocatechuate 3,4-dioxygenase gene; transformants obtained by introducing the recombinant plasmids into the disrupted strain; and a process for production of 3-carboxy-cis,cis-muconic acid and/or 3-carboxymuconolactone characterized by culturing the transformants in the presence of terephthalic acid.

### TECHNICAL FIELD

The present invention relates to a protocatechuate 4,5-ring-cleaving enzyme gene-disrupted strain, to recombinant plasmids comprising a gene coding for an enzyme participating in a multistage reaction process for fermentative production of 3-carboxy-cis,cis-muconic acid and/or 3-carboxymuconolactone from terephthalic acid via protocatechuic acid, to transformants incorporating the recombinant plasmid in the disrupted strain, and to process for industrial production of 3-carboxy-cis,cis-muconic acid and/or 3-carboxymuconolactone using the same.

### BACKGROUND ART

Terephthalic acid is an aromatic compound separated from petroleum components, and it is cheaply mass-produced as a starting material for PET. Development of new biodegradable functional plastics using terephthalic acid as the starting material will allow copolymerization with petroleum-based polymer materials such as PET, to permit the development of polymer materials with excellent biodegradability.

The present inventors have found that the terephthalic acid-degrading microorganism, Comamonas sp. E6, can completely degrade terephthalic acid via 2H-pyran-2-one-4,6-dicarboxylic acid after first converting it to protocatechuic acid (Patent document 1). There have also been reported a recombinant vector comprising the genes coding for terephthalate dioxygenase (TPA-DOX), 1,2-dihydroxy-3,5-cyclohexadiene-1,4-dicarboxylate dehydrogenase (DCD-dehydrogenase), terephthalate transporter (TPA transporter) and a positive regulator, by removing the genes coding for protocatechuate 4,5-dioxygenase and 4-carboxy-2-hydroxy-6-semialdehyde muconate dehydrogenase from the chromosomal DNA of the microorganism, transformants containing the vector, and a method of producing 2H-pyran-2-one-4,6-dicarboxylic acid from terephthalic acid using the transformants (Japanese Patent Application No. 2005-298242).

The present inventors have also reported a process for fermentative production of 3-carboxy-cis,cis-muconic acid and/or 3-carboxymuconolactone from plant components such as vanillin, vanillic acid and protocatechuic acid, via a multistage enzyme reaction (Japanese Patent Application No. 2006-218524).

In the process, terephthalic acid is converted to 3-carboxy-cis and cis-muconic acid via 1,2-dihydroxy-3,5-cyclohexadiene-1,4-dicarboxylate and further via protocatechuic acid, and the 2,3-bond, 3,4-bond or 4,5-bond of protocatechuic acid is cleaved, depending on the type of microorganism (hereunder, 2,3-bond cleavage, 3,4-bond cleavage and 4,5-bond cleavage of protocatechuic acid will be referred to as “2,3-ring cleavage”, “3,4-ring cleavage” and “4,5-ring cleavage”, respectively). In the presence of certain microorganisms, the 3-carboxy-cis,cis-muconic acid as the 3,4-ring cleavage product of protocatechuic acid is further catabolized via 3-carboxymuconolactone or 4-carboxymuconolactone.

No process has been known to date for fermentative production of 3-carboxy-cis,cis-muconic acid using terephthalic acid as the starting material.

### DISCLOSURE OF THE INVENTION

It is an object of the present invention to provide a process for industrial-scale fermentative production of 3-carboxy-cis,cis-muconic acid from terephthalic acid via protocatechuic acid, and/or a process for fermentative production of 3-carboxy-cis,cis-muconic acid from terephthalic acid via protocatechuic acid and acid treatment thereof to obtain 3-carboxymuconolactone on an industrial scale.

In order to obtain 3-carboxy-cis,cis-muconic acid efficiently, it is necessary to disrupt genes having 2,3-ring cleavage function, 3,4-ring cleavage function or 4,5-ring cleavage function, or to disrupt genes that further metabolize 3-carboxy-cis,cis-muconic acid.

The present inventors have conducted ardent research on this subject, and as a result have considered that disrupting the cleavage activity of protocatechuate 4,5-ring-cleaving enzyme would completely disrupt the conversion process from protocatechuic acid to 2H-pyran-2-one-4,6-dicarboxylic acid, and have therefore generated protocatechuate 4,5-ring-cleaving enzyme gene-disrupted strains in which the cleavage activity of protocatechuate 4,5-ring-cleaving enzyme has been disrupted. It was further found that it is possible, using the disrupted strains, to produce 3-carboxy-cis,cis-muconic acid and/or its acid treatment product, 3-carboxymuconolactone, from terephthalic acid at high yield and inexpensively.

Specifically, (1) the present invention provides a protocatechuate 4,5-ring-cleaving enzyme gene-disrupted strain in which the gene coding for:

(a) the amino acid sequence set forth in SEQ ID NO: 1 or 3, or
(b) the amino acid sequence set forth in SEQ ID NO: 1 or 3 which has a deletion, substitution, addition and/or insertion of one or more amino acids and exhibits protocatechuate 4,5-ring cleavage activity, present in the chromosomal DNA of microbial cells, has been disrupted.
(2) The present invention further provides a protocatechuate 4,5-ring-cleaving enzyme gene-disrupted strain in which a gene that comprises:
(a) the nucleotide sequence set forth in SEQ ID NO: 2 or 4; or
(b) a nucleotide sequence hybridizing with DNA consisting of a nucleotide sequence complementary to the nucleotide sequence of (a), under stringent conditions, and coding for an enzyme with protocatechuate 4,5-ring cleavage activity,
present in the chromosomal DNA of microbial cells, has been disrupted.
(3) The invention further provides a gene-disrupted strain according to (1) or (2), in which the protocatechuate 4,5-ring-cleaving enzyme gene has been disrupted by homologous recombination between a gene coding for:
(a) the amino acid sequence set forth in SEQ ID NO: 1 or 3, or
(b) the amino acid sequence set forth in SEQ ID NO: 1 or 3 which has a deletion, substitution, addition and/or insertion of one or more amino acids and exhibits protocatechuate 4,5-ring cleavage activity, present in the chromosomal DNA of microbial cells, and homologous recombination DNA having a DNA sequence that can undergo homologous recombination with the gene and lacking protocatechuate 4,5-ring cleavage activity.
(4) The invention further provides a gene-disrupted strain according to any one of (1) —(3), wherein the parent strain of the protocatechuate 4,5-ring-cleaving enzyme gene-disrupted strain is a Comamonas sp. bacterium.
(5) The invention further provides a gene-disrupted strain according to (4), wherein the Comamonas bacterium is Comamonas sp. E6.
(6) The invention further provides a recombinant plasmid comprising a terephthalate dioxygenase gene (TPA-DOX gene), NADPH-reductase gene, 1,2-dihydroxy-3,5-cyclohexadiene-1,4-dicarboxylate dehydrogenase gene (DCD dehydrogenase gene), positive regulator gene, terephthalate transporter gene (TPA transporter gene) and protocatechuate 3,4-dioxygenase gene (pcaHG gene).
(7) The invention further provides a transformant obtained by introducing a recombinant plasmid according to (6) into a gene-disrupted strain according to any one of (1)-(5).
(8) The invention further provides a process for production of 3-carboxy-cis,cis-muconic acid and/or 3-carboxymuconolactone, characterized by culturing a transformant according to (7) in the presence of terephthalic acid.

According to the invention it is possible to accomplish high-yield and inexpensive fermentative production of 3-carboxy-cis,cis-muconic acid and/or 3-carboxymuconolactone from terephthalic acid.

### BRIEF DESCRIPTION OF THE DRAWINGS

FIG. 1 is a 5.1-kbSalI-XhoI restriction enzyme and ORF map.

FIG. 2 shows the positional relationship between the pCS18 10-kb Sad fragment and the pPV10 10-kb SalI fragment.

FIG. 3 shows the protocatechuate 4,5-cleavage enzyme gene group from Comamonas sp. E6.

FIG. 4 shows the pmdB1 disrupted strain plasmid pDBKM.

FIG. 5 shows disruption of the pmdB1 gene in Comamonas sp. E6. 5(A) is an illustration of a method of constructing an E6 pmdB1 gene-disrupted strain, and 5(B) shows the results of Southern hybridization analysis of the pmdB1 gene-disrupted strain.

FIG. 6 is an illustration showing construction of the recombinant plasmid pKHG.

FIG. 7 is an illustration showing construction of the recombinant plasmid pKHG/C.

FIG. 8 is an illustration showing construction of the recombinant plasmid pKTphHG/C.

### BEST MODE FOR CARRYING OUT THE INVENTION

The gene-disrupted strain of the invention may be one in which at least one function of the 2,3-ring cleavage function, 3,4-ring cleavage function and 4,5-ring cleavage function is disrupted. Alternatively, the gene-disrupted strain of the invention may retain the 3,4-ring cleavage function, in which case the function of further metabolizing 3-carboxy-cis,cis-muconic acid must be disrupted. In order to disrupt the activity of metabolizing carboxy-cis,cis-muconic acid, the 3-carboxymuconolactonizing enzyme or 4-carboxymuconolactonizing enzyme may be disrupted.

Production of 3-carboxy-cis,cis-muconic acid according to the invention is accomplished by introducing the gene-disrupted strain of the invention into, for example, i) a host microorganism having a function of metabolizing terephthalic acid to protocatechuic acid (terephthalate assimilation), and lacking a terephthalate 2,3-ring cleavage function but having a terephthalic acid 3,4-ring cleavage function or 4,5-ring cleavage function, or ii) a host microorganism lacking terephthalate assimilation but having “protocatechuate assimilation” by a protocatechuate 2,3-ring cleavage function, 3,4-ring cleavage function or 4,5-ring cleavage function.

The invention will now be explained in detail using a gene-disrupted strain with disrupted protocatechuate 4,5-ring cleavage function as an example.

(I) Obtaining Gene Coding For Protocatechuate 4,5-Ring-Cleaving Enzyme (Protocatechuate 4,5-Dioxygenase)

For the purpose of the invention, the protocatechuate 4,5-ring cleavage enzyme gene group will be referred to as “pmd gene group.” Of the pmd gene group, the gene coding for the α-subunit of the enzyme having dioxygenase activity that cleaves the protocatechuate 4,5-ring for conversion to 4-carboxy-2-hydroxy-6-semialdehyde muconate will be referred to as “pmdA1” (the amino acid sequence set forth in SEQ ID NO: 3 and the nucleotide sequence set forth in SEQ ID NO: 4), and the gene coding for the β-subunit of the same will be referred to as “pmdB1” (the amino acid sequence set forth in SEQ ID NO: 1 and the nucleotide sequence set forth in SEQ ID NO: 2).

Also, the gene coding for the enzyme with dehydrogenase activity that cleaves 4-carboxy-2-hydroxy-6-semialdehyde muconate for conversion to 2H-pyran-2-one-4,6-dicarboxylic acid will be referred to as “pmdC”.

The method of obtaining the gene coding for protocatechuate 4,5-ring-cleaving enzyme is not particularly restricted, and for example, the gene may be obtained by preparing a suitable probe or primer based on the data for the nucleotide sequence of the gene, and screening a cDNA library or genomic DNA library for the strain, using the probe or primer.

The gene for protocatechuate 4,5-ring-cleaving enzyme may also be obtained by PCR. The chromosomal DNA or cDNA library of the strain may be used as template for PCR with a pair of primers designed so as to amplify the nucleotide sequence of the gene. The PCR reaction conditions may be set as appropriate, and for example, it may be carried out under conditions in which reaction for 30 seconds at 94° C. (denaturation), 30 seconds to 1 minute at 55° C. (annealing) and 2 minutes at 72° C. (extension) as one cycle, is carried out for 30 cycles followed by reaction for 7 minutes at 72° C. The amplified DNA fragment may then be cloned in a suitable vector. The vector is preferably selected as a vector that is autoreplicating in E. coli and that is incapable of extrachromosomal autoreplication in Comamonas sp.

The procedures for preparation of the probe or primer, construction of the cDNA library, screening of the cDNA library and cloning of the target gene may be known to those skilled in the art, and for example, they may be carried out according to the methods described in Molecular Cloning: A laboratory Manual, 2nd Edition, Cold Spring Harbor Laboratory, Cold Spring Harbor, N.Y., 1989 and Current Protocols in Molecular Biology, Supplement pp. 1-38, John Wiley & Sons (1987-1997).

(II) Construction of Gene-Disrupted Strain

The gene-disrupted strain of the invention is a disrupted strain wherein protocatechuate 4,5-ring cleavage has been selectively disrupted. More specifically, the gene coding for: (a) the amino acid sequence set forth in SEQ ID NO: 1 or 3, or (b) the amino acid sequence set forth in SEQ ID NO: 1 or 3 which has a deletion, substitution, addition and/or insertion of one or more amino acids and exhibits protocatechuate 4,5-ring cleavage activity, present in the chromosomal DNA of microbial cells, has been disrupted.

The gene may be a gene that comprises (a) the nucleotide sequence set forth in SEQ ID NO: 2 or 4; or (b) a nucleotide sequence hybridizing with DNA consisting of a nucleotide sequence complementary to the nucleotide sequence of (a) under stringent conditions, and coding for an enzyme with protocatechuate 4,5-ring cleavage activity.

Specifically, the gene to be disrupted may be the pmdB1 gene set forth in SEQ ID NO: 2, the pmdA1 gene set forth in s SEQ ID NO: 4, or both of these genes.

There are no particular restrictions on the range of “one or more” in the phrase “amino acid sequence having a deletion, substitution, addition and/or insertion of one or more amino acids” used throughout the present specification, and it may be, for example, 1-20, preferably 1-10, more preferably 1-7 and most preferably about 1-3.

The phrase “stringent hybridization conditions” used in the present specification means “highly stringent conditions” under which a DNA chain can hybridize to another DNA chain which is highly complementary to the DNA chain, and which is designed so as to exclude significantly mismatched DNA hybridization, or “moderately stringent conditions” under which DNA double strands can form with a greater degree of base pair mismatching than is possible under “highly stringent conditions”. As specific examples of “highly stringent conditions” there may be mentioned 0.015 M sodium chloride and 0.0015 M sodium citrate at 65-68° C., or 0.015 M sodium chloride, 0.0015 M sodium citrate, and 50% formamide at 42° C. As specific examples of “moderately stringent conditions” there may be mentioned 0.015 M sodium chloride and 0.0015 M sodium citrate at 50-65° C., or 0.015 M sodium chloride, 0.0015 M sodium citrate, and 20% formamide at 37-50° C.

As specific hybridizing DNA there may be mentioned DNA having at least 60%, preferably at least 70%, more preferably at least 80%, even more preferably at least 90%, yet more preferably at least 95% and most preferably at least 97% homology with a polynucleotide containing the nucleotide sequence set forth in SEQ ID NO: 2 or 4, as calculated using analysis software such as BLAST [J. Mol. Biol., 215, 403 (1990)] or FASTA [Methods in Enzymology, 183, 63 (1990)].

Throughout the present specification, “having protocatechuate 4,5-ring cleavage activity” means activity equivalent to an enzyme that cleaves the protocatechuate 4,5-ring to convert protocatechuic acid to 4-carboxy-2-hydroxy-6-semialdehyde muconate.

The gene-disrupted strain of the invention can be generated by introducing a mutation and/or a selective marker into the gene coding for protocatechuate 4,5-ring-cleaving enzyme obtained by PCR or cloning, to obtain DNA lacking protocatechuate 4,5-ring cleavage activity, and then using the DNA for homologous recombination.

The method for introducing a selective marker into the gene coding for protocatechuate 4,5-ring-cleaving enzyme may be, for example, a method in which the gene is cut with an appropriate restriction enzyme and then an unrelated gene, preferably a selective marker gene that allows selection of strains that have undergone homologous recombination, is inserted. When no suitable restriction enzyme site is present, a suitable restriction enzyme site may be introduced by PCR or the like. As selective markers there are preferred drug resistance markers, and as examples there may be mentioned kanamycin resistance genes, ampicillin resistance genes and tetracycline resistance genes.

The method for introducing mutations into the gene coding for protocatechuate 4,5-ring-cleaving enzyme may be any of the numerous known methods, such as recombinant DNA techniques for manipulating DNA nucleotide sequences (Sambruck, J., Fritsch, E. F. and Maniatis, T. (1989) Molecular Cloning: A Laboratory Manual, Cold Spring Harbor Laboratory, Cold Spring Harbor, N.Y.), and PCR-applied techniques (Ling M. M. and Robinson B H., Anal. Biochem. 254(2): 157-78, 1997).

The DNA lacking protocatechuate 4,5-ring cleavage activity, used to generate the gene-disrupted strain of the invention, may be any that undergoes homologous recombination with a gene coding for the corresponding protocatechuate 4,5-ring-cleaving enzyme on the chromosome under physiological conditions, i.e. in microbial cells, having sufficient homology to thus allow disruption of the protocatechuate 4,5-ring-cleaving enzyme gene. The homology is preferably 80% or greater, more preferably 90% or greater and most preferably 95% or greater. The DNA used for homologous recombination may also be a portion of the protocatechuate 4,5-ring-cleaving enzyme gene, so long as it undergoes homologous recombination with a gene coding for the corresponding protocatechuate 4,5-ring-cleaving enzyme on the chromosome under physiological conditions, i.e. in microbial cells, thereby allowing disruption of the protocatechuate 4,5-ring-cleaving enzyme gene. The portion referred to here may be a length of preferably 50 or more nucleotides, and more preferably 100 or more nucleotides.

In order to generate a gene-disrupted strain by homologous recombination, first there is constructed DNA containing the nucleotide sequence of the protocatechuate 4,5-ring-cleaving enzyme gene or recombinant DNA comprising the mutated DNA having an appropriate selective marker inserted therein. When a drug resistance marker is used as the selective marker, it is necessary to select a resistance gene for a drug to which the wild type strain prior to homologous recombination is sensitive. This will allow discernment between strains that have undergone homologous recombination and strains that have not, based on growth in the presence of an antibiotic. Next the DNA having the selective marker inserted into the gene sequence is introduced into the strain by electroporation or the like, and then selection is carried out using the marker to incorporate the target gene into the host microorganism chromosomes by homologous recombination.

The parent strain of the protocatechuate 4,5-ring-cleaving enzyme gene-disrupted strain is not particularly restricted so long as it is a soil bacterium of Comamonas, Pseudomonas, Bacillus, Lactobacillus, Streptococcus, Saccharomyces, Candida or the like. The parent strain may be a strain derived from a microorganism that is capable of “terephthalate assimilation”, or a strain derived from a microorganism that is incapable of “terephthalate assimilation” but is capable of “protocatechuate assimilation”.

A microorganism that is capable of terephthalate assimilation is able to metabolize terephthalic acid to protocatechuic acid. As examples of strains capable of terephthalate assimilation, there may be mentioned Comamonas sp. E6, Pseudomonas putida PPY1100, Comamonas testosteroni (C. testosteroni) T-2, C. testosteroni YZW-D, Derftia tsuruhatensis T7, Rhodococcus sp. DK17 and Rhodococcus jostii RHA1. Of these, Comamonas sp. E6 is a strain that retains ability to metabolize terephthalic acid to protocatechuic acid and ability to metabolize protocatechuic acid to 2H-pyran-2-one-4,6-dicarboxylic acid (4,5-ring cleavage function), but does not retain 2,3-ring cleavage function and 3,4-ring cleavage function. When Comamonas sp. E6 was used as the parent strain, the production efficiency for 3-carboxy-cis,cis-muconic acid was roughly equivalent to a fermentative production system from vanillic acid to 2H-pyran-2-one-4,6-dicarboxylic acid (Japanese Unexamined Patent Publication No. 2005-278549) or a fermentative production system to 3-carboxy-cis,cis-muconic acid and/or 3-carboxymuconolactone (Japanese Patent Application No. 200-218524).

A microorganism that is not capable of terephthalate assimilation but is capable of protocatechuate assimilation has a 2,3-ring cleavage function, 3,4-ring cleavage function or 4,5-ring cleavage function. As examples of such microorganisms there may be mentioned microorganisms belonging to the genus Pseudomonas, Bacillus, Burkholderia and Agrobacterium.

A gene-disrupted strain with disrupted protocatechuate 2,3-ring cleavage function or 3,4-ring cleavage function can also be generated in the same manner as the aforementioned 4,5-ring cleavage function-disrupted strain.

A microorganism capable of terephthalate assimilation will generally have more developed terephthalate uptake and metabolism compared to a microorganism incapable of terephthalate assimilation, and higher conversion efficiency would be expected.

Incidentally, 3-carboxy-cis,cis-muconic acid can also be efficiently produced using a strain that is not a gene-deleted strain according to the invention but which retains protocatechuate 3,4-ring cleavage function and does not further catabolize the 3-carboxy-cis,cis-muconic acid cleavage product. Pseudomonas putida PPY1100 may be mentioned as an example of such a strain, but the reaction efficiency is slightly lower with Pseudomonas putida PPY1100 compared to Comamonas sp. E6.

For even more efficient production of 3-carboxy-cis,cis-muconic acid, it is more preferred to use a gene-disrupted strain having disrupted protocatechuate 2,3- and 4,5-ring cleavage activity, and also having disrupted 3-carboxy-cis,cis-muconic acid metabolism activity in order to completely eliminate the possibility of further catabolism of 3-carboxy-cis,cis-muconic acid.

(III) Preparation of Transformants Incorporating 3-Carboxy-Cis,Cis-Muconic Acid Fermentative Production Plasmid

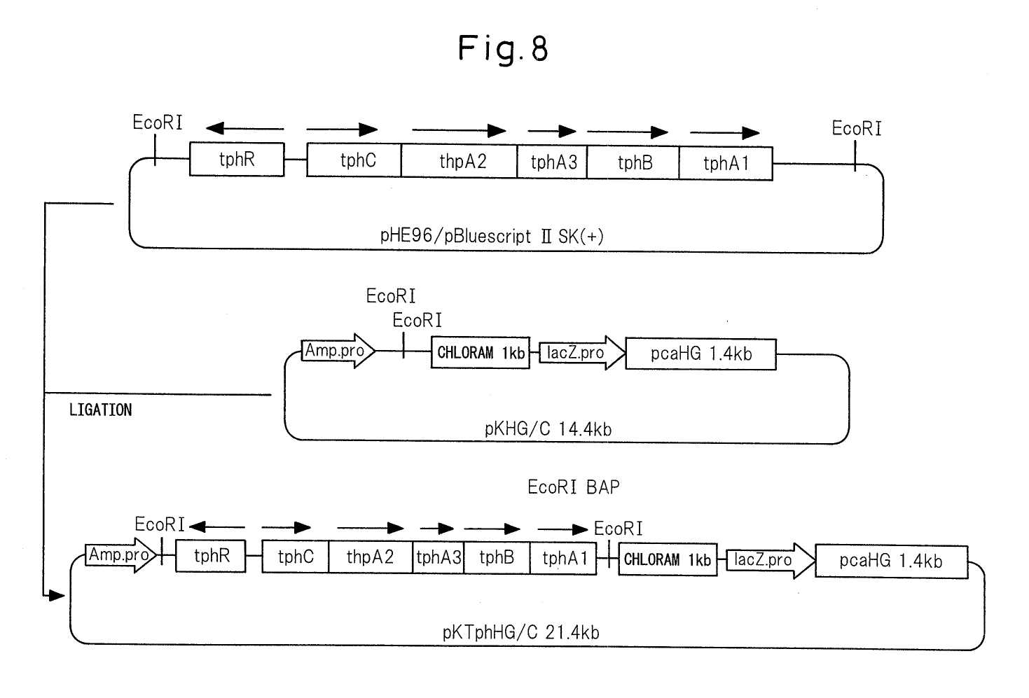

The plasmid is a plasmid comprising genes for enzymes that catalyze a multistage process for production of 3-carboxy-cis,cis-muconic acid from terephthalic acid via protocatechuic acid (positive regulator, TPA-transporter, TPA-DOX, DCD-dehydrogenase, NADPH-reductase, protocatechuate 3,4-ring cleavage gene), in that order from the upstream end (FIG. 8).

The positive regulator gene (tphR) is the DNA molecule set forth in SEQ ID NO: 11 in Japanese Patent Application No. 2005-298242 (SEQ ID NO: 9 in the present specification), the TPA-transporter gene (tphC) is the DNA molecule set forth in SEQ ID NO: 13 in the same specification (SEQ ID NO: 10 in the present specification), the TPA-DOX genes (tphA2, tphA3) are the DNA molecules set forth in SEQ ID NO: 2 and 4 in the same specification (SEQ ID NO: 11 and 12, respectively, in the present specification), the DCD-dehydrogenase gene (tphB) is the DNA molecule set forth in SEQ ID NO: 6 in the same specification (SEQ ID NO: 13 in the present specification), and the NADPH-reductase gene (tphA1) is the DNA molecule set forth in SEQ ID NO: 8 in the same specification (SEQ ID NO: 14 in the present specification). The protocatechuate 3,4-ring cleavage genes (pcaH and G) used for the invention are DNA fragments obtained from Pseudomonas putida KT2440, and the nucleotide sequence of the PcaH gene is set forth in SEQ ID NO: 1 of Japanese Patent Application No. 2006-218524 while the nucleotide sequence of the PcaG gene is set forth in SEQ ID NO: 3 of the same specification (SEQ ID NO: 15 and 16 of the present specification). In the present specification, tphR, tphC, tphA2, tphA3, tphB and tphA1 will also be collectively referred to as “Tph gene cluster” or “Tph gene group”.

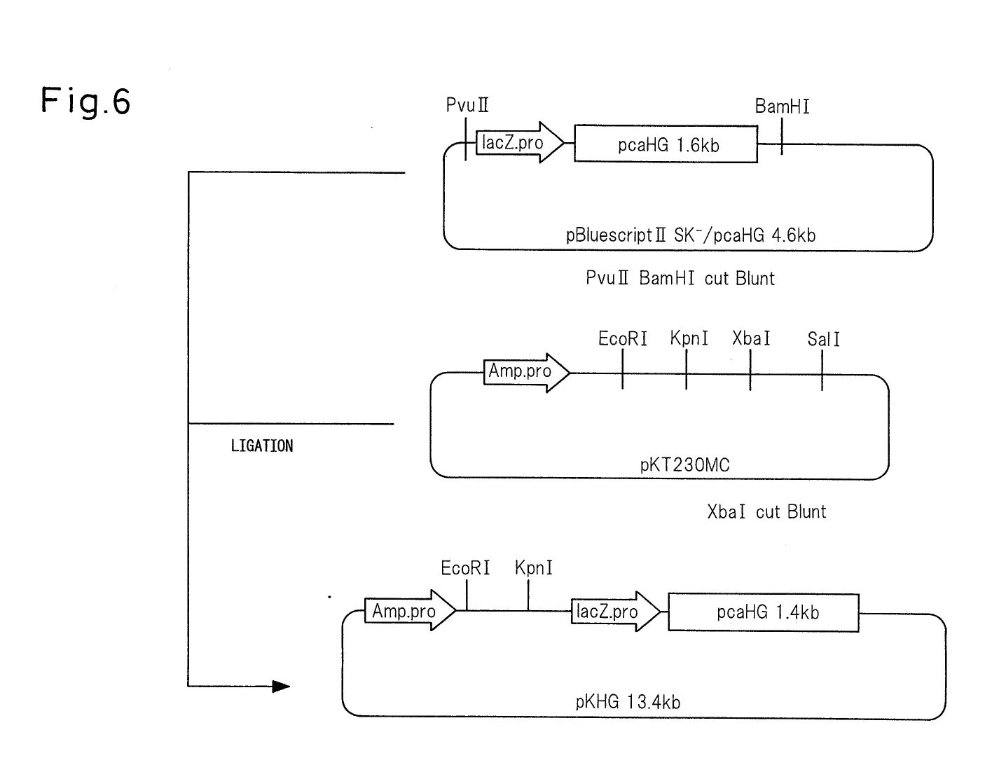

Specifically, the plasmid for fermentative production of 3-carboxy-cis,cis-muconic acid from terephthalic acid according to the invention may be constructed as illustrated in FIGS. 6 to 8.

(1) First, the PcaH gene (SEQ ID NO: 1) and PcaG gene (SEQ ID NO: 2) listed in Japanese Patent Application No. 2006-218524 are ligated to a multicloning site in the gene coding for the α-fragment of LacZ, present downstream from the pBluescript LacZ promoter, using a known ligase, to construct recombinant plasmid pBluescript II SK⁻/pcaHG.

Next, a known ligase is used to ligate a DNA fragment obtained by end treatment after cutting pBluescript II SK⁻/pcaHG with restriction enzymes PvuII and BamHI, with a DNA fragment obtained by end treatment after cutting an Amp promoter-containing plasmid with restriction enzyme XbaI, to construct recombinant plasmid pKHG (FIG. 6).

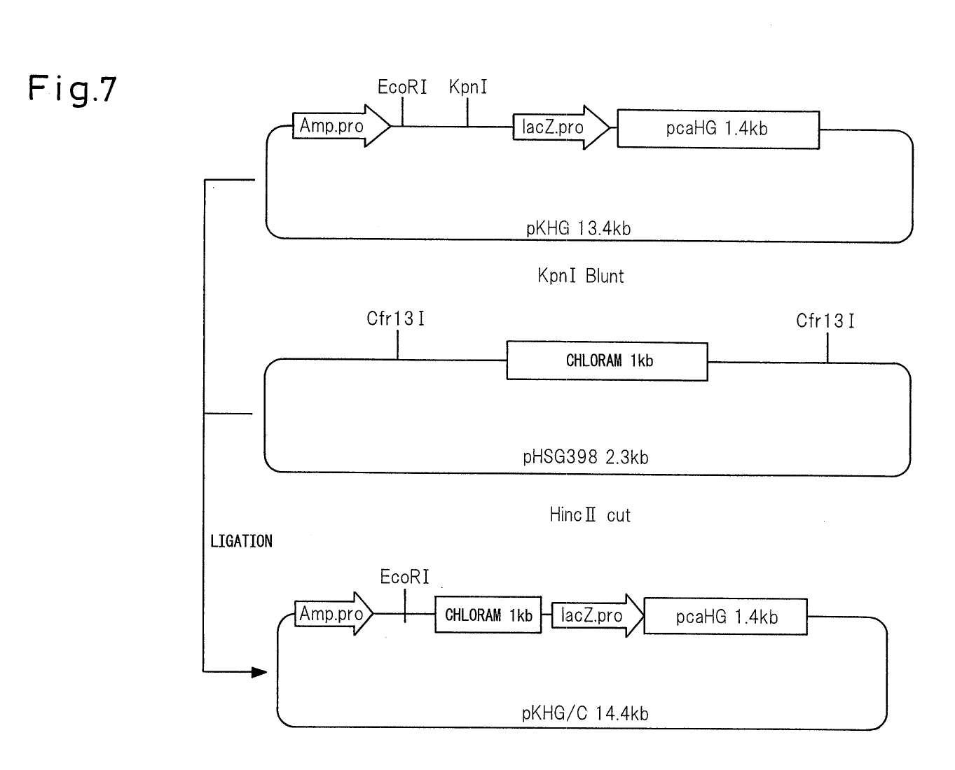

(2) Next, a known ligase is used to ligate a DNA fragment obtained by end treatment after cutting a chloramphenicol resistance gene-containing plasmid with restriction enzyme Cfr13I, with a DNA fragment obtained by end treatment after cutting pKHG with restriction enzyme KpnI, to construct recombinant plasmid pKHG/C (FIG. 7).

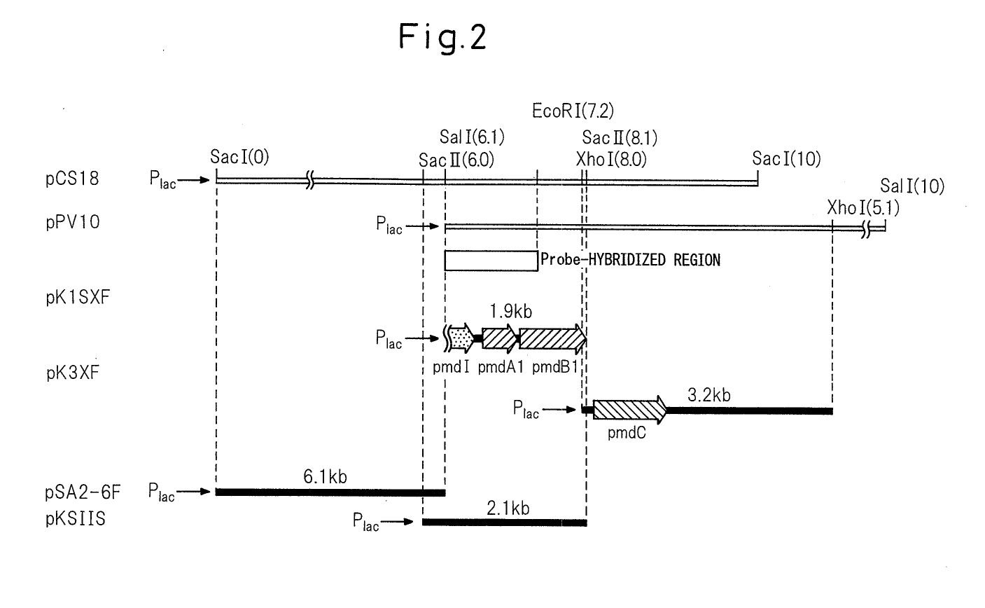

(3) Also, a known ligase may be used to ligate a DNA fragment obtained by cutting recombinant plasmid pHE96/pBluescript II SK (+), having tphR, tphC, tphA2, tphA3, tphB and tphA1 ligated in that order from the upstream end and shown in FIG. 2 of Japanese Patent Application No. 2005-298242, with restriction enzyme EcoRI, with a DNA fragment obtained by end treatment after cutting the pKHG/C obtained in (2) with EcoRI, to construct recombinant plasmid pKTphHG/C.

A known method such as a protoplast method, competent cell method or electroporation method may be used for transformation of the gene-disrupted strain of (I) using the recombinant plasmid pKTphHG/C.

Selection of transformants may be accomplished based on a selective marker for the plasmid used, such as drug resistance acquired by DNA recombination in the transformants. The transformants containing the recombinant plasmid of interest are preferably selected from among the transformants by colony hybridization using a partial DNA fragment of the gene as the probe. Labeling of the probe may be carried out using a radioactive isotope, digoxigenin, an enzyme or the like.

(IV) The Fermentative Production of 3-Carboxy-Cis,Cis-Muconic Acid and/or 3-Carboxymuconolactone

The transformants of the invention of (II) may be cultured under appropriate conditions in the presence of terephthalic acid, using medium containing a carbon source, nitrogen source, metal salts, minerals, vitamins and the like. The pH of the medium may be a pH in a range that allows growth of the transformants, and the pH is preferably adjusted to about 6-8. The culturing conditions may be shake culturing or submerged culturing for 2-7 days at 15-40° C. and preferably 28-37° C.

Ordinary isolation and purification methods for organic compounds may be used for isolation and purification of 3-carboxy-cis,cis-muconic acid from the cultured gene-disrupted strain. For example, upon completion of culturing, the cells are collected by centrifugal separation and suspended in aqueous buffer, and then disrupted using an ultrasonic disruptor or the like to obtain a cell-free extract. The target substance may be obtained by ordinary isolation and purification methods for organic compounds, from the supernatant obtained by centrifugal separation of the cell-free extract.

Acid treatment of the 3-carboxy-cis,cis-3-muconic acid obtained in this manner, or the culture solution containing the unpurified 3-carboxy-cis,cis-3-muconic acid, will allow conversion to 3-carboxymuconolactone at a high yield. The acid used is preferably hydrochloric acid at about pH 1-2.

The 3-carboxy-cis,cis-muconic acid and/or 3-carboxymuconolactone obtained by the production process of the invention, as a plastic material, chemical product material or the like, can exhibit functions different from 2H-pyran-2-one-4,6-dicarboxylic acid or higher functions thereof, and therefore can serve as a useful plastic material.

### EXAMPLES

The present invention will now be described in greater detail by examples, with the understanding that the invention is not limited to these examples.

### Example 1

### Cloning of pmd Gene Group

### (1) Amplification of Protocatechuate 4,5-Dioxygenase Gene by PCR and Sequencing

A region of high homology was identified from alignment of the amino acid sequences deduced from the ligA gene of Sphingomonas paucimobilis SYK-6, the pcmA gene of Arthrobacter keyseri 12B, the pmdA1B1 gene of Comamonas testosteroni BR6020 and the fldVU gene of Sphingomonas sp. LB126, and PCR was conducted using the following αF/βR primer, with the total DNA of the Comamonas sp. E6 protocatechuate 4,5-dioxygenase gene as template.

αF of α-subunit
(26 mer:
ATGWSSCTGATGAARSCSGARAACCG; SEQ ID NO: 5)
βf of β-subunit;
(27 mer:
GTSWTCCTGGTSTAYAACGAYCAYGCY; SEQ ID NO: 6)
and
βR of β-subunit.
(25 mer:
CCCTGMARCTGRTGGCTCATGCCGC; SEQ ID NO: 7)

As a result, amplification was seen at the predicted size of 900 bp. The PCR product was then used as template for PCR between βF-βR, using the nested primer βF. As a result, amplification was seen of a 450 by fragment of the predicted size. The obtained PCR product was subcloned in pT7Blue vector to determine the nucleotide sequence of the 449 by fragment. A homologous sequence search was conducted in the DDBJ database, and the gene coding for the Comamonas testosteroni BR6020 protocatechuate 4,5-dioxygenase β-subunit (DDBJ Accession No.: AF305325) showed 99% homology on the amino acid level. Also, homology of 68% was found with Sphingomonas paucimobilis SYK-6 ligB (DDBJ Accession No.: AB035122), and 66% with both Arthrobacter keyseri 12B pcmA (DDBJ Accession No.: AF331043) and Sphingomonas sp. LB126 fldU (DDBJ Accession No.: AJ277295).

### (2) Isolation Of Cosmid Containing Protocatechuate 4,5-Dioxygenase Gene

Positive clones were obtained from a cosmid library containing E6 SalI partial digestion fragments, by colony hybridization using the PCR product as the probe. The cosmid extracted from the obtained positive clones was digested with SalI and subjected to Southern hybridization with the same probe, which produced hybridization of 10 kb, and the cosmid was named pPV10.

(3) Nucleotide Sequences of pPV10-Derived 1.9-Kb SalI-XhoI Fragment and 3.2-kb XhoI Fragment

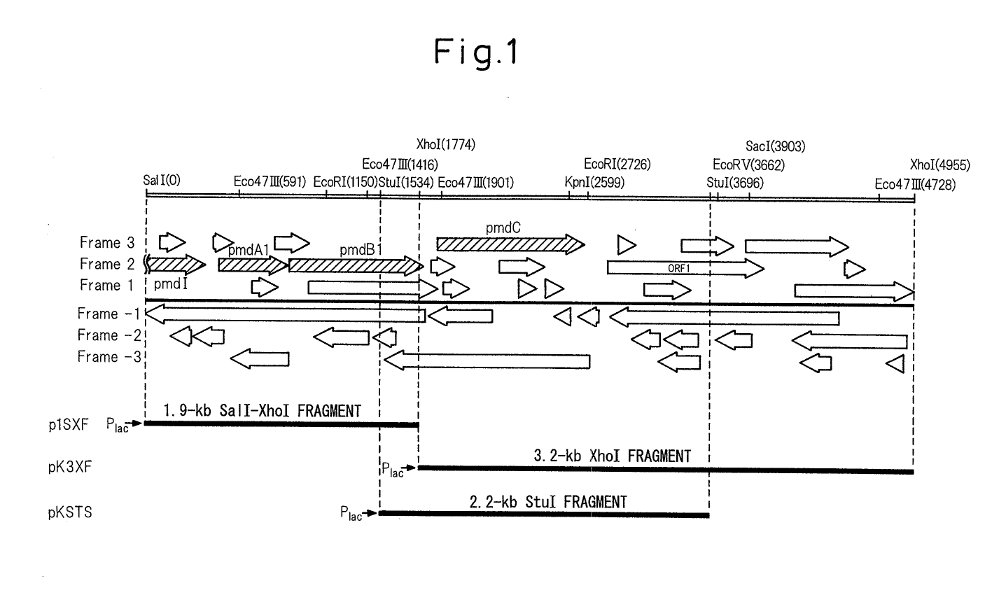

Clone pKS10F(R) was constructed by reciprocal bidirectional insertion of the pPV10 10-kb SalI fragment downstream from the pBluescript II KS (+) lac promoter. Three fragments of 1.9 kb, 3.2 kb and 4.9 kb, obtained by digestion of pKS10F with SalI and XhoI, were blunted and inserted into the SmaI site of pBluescript II KS(+). After reciprocal bidirectional insertion of these fragments downstream from the lac promoter, the obtained plasmids were named pK1SXF(R), pK3XF(R) and pK4XSF(R), respectively (FIG. 1). The full nucleotide sequences of the pK1SXF(R) 1.9-kb SalI-XhoI fragment and the 3.2-kb XhoI fragment in pK3X were determined. Sequencing of the pKSTS 2.2-kb StuI fragment also confirmed that these fragments are adjacent across XhoI (FIG. 1). In FIG. 1, Plac represents the lac promoter, with the arrow indicating the direction of transcription.

(4) Isolation of Region Upstream from 10-Kb SalI Fragment

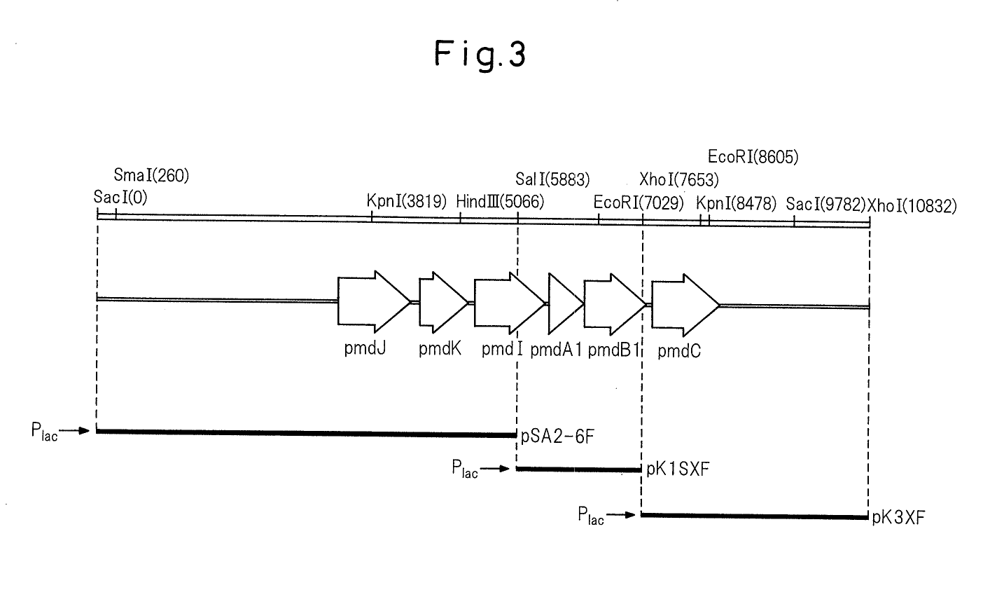

In order to isolate the region upstream from the pPV10 10-kb SalI fragment, a library was prepared by cloning of a Sad digest of E6 total DNA in charomid 9-36. A 1,150 by fragment obtained by digestion of the 1.9-kb SalI-XhoI fragment with SalI and EcoRI was used for colony hybridization, to isolate pCS18 having a 10-kb SacI fragment containing pmdA1B1 (FIG. 2). After nucleotide sequencing to confirm that the pSC18 2.1-kb SacII fragment containing pmdA1B1 is adjacent to the 6.1-kb SacI-SalI fragment, clone pSA2-6F(R) obtained by blunting the 6.1-kb SacI-SalI fragment and reciprocal bidirectional insertion at the EcoRV site downstream from the pBluescript II KS(+) lac promoter was generated, and the full nucleotide sequence was determined (FIG. 3). SEQ ID NO: 8 includes the gene sequence comprising the genes pmdJ (base numbers 1-1029), pmdK (base numbers 1162-1845), pmdI (base numbers 1942-2934), pmdA1 (SEQ ID NO: 2), pmdB1 (SEQ ID NO: 4 and pmdC (base numbers 4427-5386), shown in FIG. 3.

### Example 2

### Construction of pmdB1 Gene-Disrupted Strain

(1) Preparation of pmdB1 Gene-Disrupting Plasmid

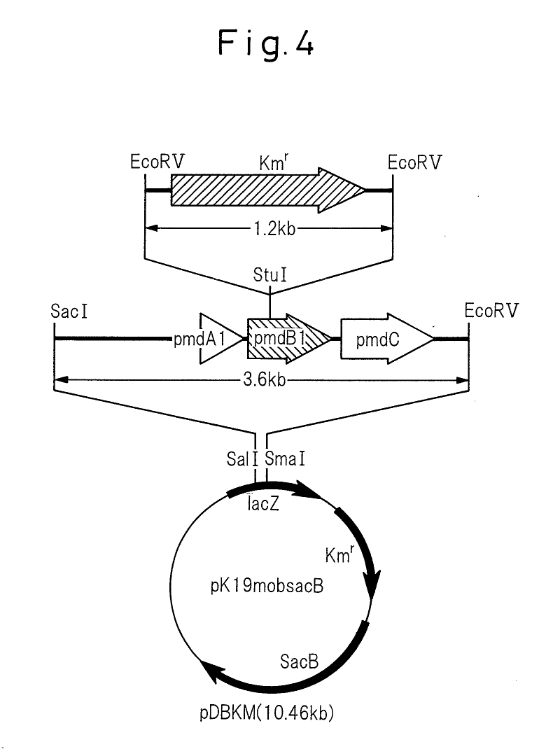

A 3.6-kb SalI-EcoRV fragment containing the pmdA1B1C gene was cut out from pKS10F and inserted into a SalI-EcoRV digest of pBluescript II KS(+) to obtain pSDB36. It was then digested with StuI within the pmdB1 gene, and a 1.2-kb EcoRV fragment containing the pIK03-derived kanamycin resistance gene was inserted therein (the underlined portion of the sequence of SEQ ID NO: 1). Insertion of the fragment in the same direction as the pBluescript II KS(+) lac promoter transcription direction was confirmed by SmaI digestion, thus obtaining pKS78F. A 4.9-kb SalI-EcoRV fragment was cut out from pKS78F and inserted at the SalI-SmaI site of pK19 mobsacB to construct plasmid pDBKM for generated of a pmdB1-disrupted strain (FIG. 4).

(2) Construction of pmdB1 Gene-Disrupted Strain

E6 precultured in 3 ml of LB medium was inoculated at 1% into 10 ml of LB medium, for main culturing. After growing at OD600=0.5, the culture was centrifuged at 5,000 rpm, 4° C., 15 minutes and the cells were collected. After rinsing twice in 1 ml of 0.3 M sucrose, it was suspended in 1 ml of 0.5 M sucrose. Next, 1 μl of the gene-disrupting plasmid pDBKM prepared to 1 μg/μl was added to 100 μl of the culture and subjected to pulsing using a Gene pulser (Bio-Rad), under conditions with a resistance of 800Ω, a voltage of 12 V and an electrostatic capacity of 25 μF. Immediately after pulsing, 1 ml of LB medium was added and culturing was carried out at 30° C. for 6 hours. A 300 μl portion thereof was coated onto 100 μg/ml kanamycin-containing LB medium.

The obtained kanamycin-resistant strains were collected with 1 ml of LB medium, seeded in 10 ml of LB medium containing 10% sucrose, and cultured for 12 hours. A 200 μl portion was then transferred into fresh LB medium and the procedure was repeated three times. Finally, the culture solution was coated onto 100 μg/ml kanamycin-containing LB medium and the obtained colonies were used as candidate gene-disrupted strains.

(3) Confirmation of pmdB1 Gene Disruption

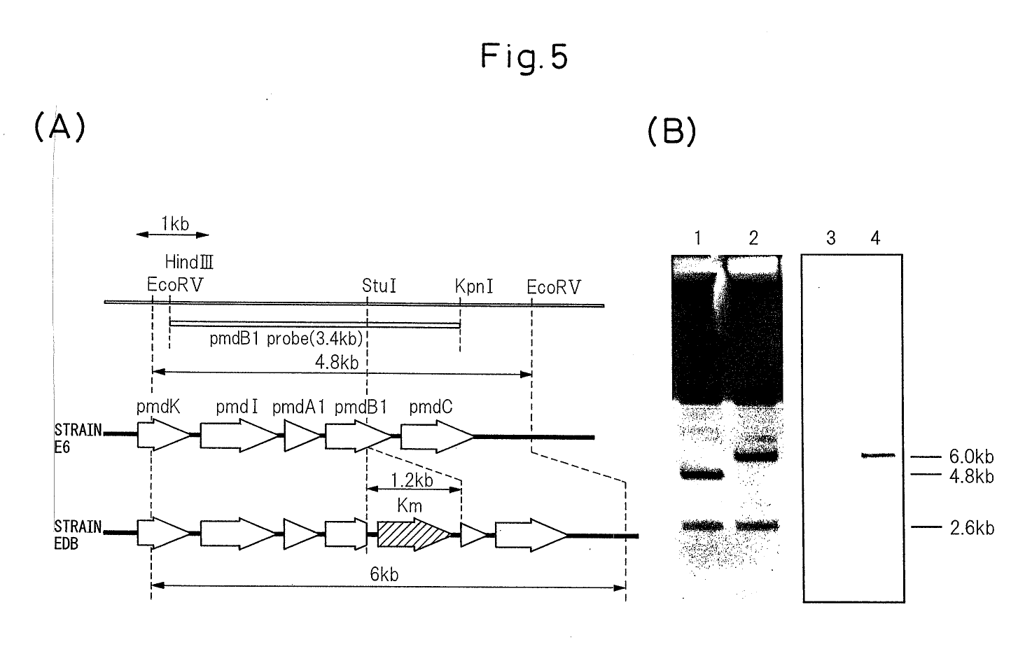

The candidate gene-disrupted strains were precultured in 3 ml of 100 μg/ml kanamycin-containing LB medium, and inoculated at 1% into 10 ml of culture medium for main culturing. Next, the total DNA of the candidate gene-disrupted strains were recovered and digested with EcoRV, after which a 3.4-kb HindIII-KpnI fragment containing pmdA1B1 and a 1.2-kb EcoRV fragment containing the pIK03-derived kanamycin resistance gene were used as probes for Southern hybridization (FIG. 5). Lanes 1 and 3 represent the total DNA of the wild type E6 digested with EcoRV, and lanes 2 and 4 represent the total DNA of the gene-disrupted strain digested with EcoRV. The probe used was a pmdB1-containing 3,4-kb HindIII-KpnI fragment for lanes 1 and 2 and a kanamycin (Km) resistance gene-containing 1.2-kb EcoRV fragment for lanes 3 and 4.

### Example 3

### Production of 3-carboxymuconolactone

(1) Construction of Recombinant Plasmid pKTphHG/C for 3-Carboxy-Cis,Cis-Muconic Acid

1-1) A DNA fragment obtained by cutting the recombinant plasmid pBluescript II SK⁻/pcaHG mentioned in Japanese Patent Application No. 2006-218524 with restriction enzymes pvuII and BamHI and then blunting the ends, and a DNA fragment obtained by cutting pKT230MC mentioned in Japanese Unexamined Patent Publication No. 2005-278549 with restriction enzyme XbaI and then blunting the ends, were ligated with T4DNA ligase (Roche) to construct recombinant plasmid pKHG (FIG. 6).

1-2) Next, a DNA fragment obtained by cutting chloramphenicol resistance gene-containing plasmid pHSG398 (product of Takara) with restriction enzyme Cfr13I and then blunting the ends, and a DNA fragment obtained by cutting pKHG with restriction enzyme KpnI and then blunting the ends, were ligated with T4DNA ligase (Roche) to construct recombinant plasmid pKHG/C (FIG. 7).

1-3) Also, a DNA fragment obtained by cutting recombinant plasmid pHE96/pBluescript II SK(+) mentioned in Japanese Patent Application No. 2006-218524 with restriction enzyme EcoRI and a DNA fragment obtained by cutting pKHG/C with restriction enzyme EcoRI were ligated with T4DNA ligase (Roche) to construct recombinant plasmid pKTphHG/C (FIG. 8).

### (2) Transformation

2-1) Recombinant plasmid pKTphHG/C was used to transform E. coli HB101, and the transformants were shake cultured at 37° C. for 18 hours in LB medium (100 ml) containing 25 mg/L kanamycin, after which the recombinant plasmid pKTphHG/C was extracted from the proliferated cultured cells.

2-2) The gene-disrupted strain generated in Example 2 (Comamonas sp. EDB) was cultured at 28° C. for 23 hours in 500 ml of LB liquid medium and cooled on ice for 30 minutes. The cells were collected by centrifugation at 4° C., 10,000 rpm for 10 minutes, and after mild rinsing with 500 ml of 0° C. distilled water, they were re-centrifuged. This was followed by additional mild rinsing with 250 ml of 0° C. distilled water and re-centrifugation. This was further followed by additional mild rinsing with 125 ml of 0° C. distilled water and re-centrifugation. The collected microbial cells were suspended in distilled water containing 10% glycerol and stored at 0° C.

2-3) After placing 4 μl of distilled water containing about 0.05 μg of the DNA of plasmid pKTphHG/C of (1) in a 0.2 cm cuvette, 40 μl of the cell solution suspended in distilled water containing 10% glycerol obtained in 2-2) above was added, and the mixture was subjected to electroporation under conditions of 25 μF, 2500 V, 12 msec.

2-4) The total amount of treated cells was seeded in 10 ml of LB liquid medium and cultured at 28° C. for 6 hours. After culturing, the cells were collected by centrifugation, developed on an LB plate containing 25 mg/L kanamycin, 50 mg/L ampicillin and 30 mg/L chloramphenicol and cultured at 28° C. for 48 hours, to obtain transformants retaining plasmid pKTphHG/C and exhibiting chloramphenicol resistance. The cells were designated as strain pKTphHG/C/Comamonas sp. ECB.

2-5) Strain pKTphHG/C/Comamonas sp. ECB was seeded in 200 ml of LB liquid medium (containing 30 mg/L chloramphenicol) and cultured at 28° C. for 16 hours, to produce a cultured cell suspension. After preparing 5 L of LB liquid medium and 3 ml of antifoaming agent (Antifoam A) using a 10 L-volume jar fermenter, an aqueous solution obtained by dissolving 200 ml of a precultured cell suspension of pKTphHG/C/Comamonas sp. ECB cultured therein and 8 g of terephthalic acid was mixed therewith, and aerated stirring was carried out at 500 rpm/min at 28° C., for culturing to OD518=approximately 3 (8 hours-12 hours).

2-6) When the OD518 reached 3 with culturing using a 10 L-volume jar fermenter, 500 ml of culture solution was removed from the fermenter into an Erlenmeyer flask and stored on ice.

2-7) To the culture solution in the fermenter that had reached OD518 of 3 there was added 42 g of terephthalic acid dissolved in 500 ml of a 0.1 N NaOH aqueous solution (adjusted to pH 8.5), over a period of 10-12 hours using a peristaltic pump. In order to prevent reduction in the pH of the culture solution with production of 3-carboxy-cis,cis-muconic acid as the reaction proceeded, a 0.1 N NaOH solution was added with a peristaltic pump connected to a pH sensor to maintain the pH of the culture solution. Progress of the reaction was confirmed by HPLC. After 36 hours, the added terephthalic acid had virtually disappeared. A 500 ml portion of the ice-cooled cell suspension prepared in 2-5) was added to the culture solution in the fermenter and culturing was continued for 12 hours.

2-8) Upon completion of the reaction, the medium in the fermenter was transferred to a plastic container (bucket). The cell component was precipitated and removed from the culture solution by centrifugal separation (6000 rpm, 20° C.), hydrochloric acid was added to the obtained supernatant to lower the pH to below 1.0, and the mixture was stored at low temperature for conversion of the 3-carboxy-cis,cis-muconic acid to 3-carboxymuconolactone. After confirming complete conversion to 3-carboxymuconolactone by GC-MS, an organic solvent (ethyl acetate) was used for extraction of the 3-carboxymuconolactone. The amount of extracted and dried 3-carboxymuconolactone reached approximately 9.8 g from 1 L of culture solution, which was a yield of about 87% as the ratio of added substrate (terephthalic acid). The obtained 3-carboxymuconolactone was further treated with active carbon and the structure was confirmed by its NMR and MS spectra.

¹H-NMR (400 MHz, DMSOd₆) δ: 2.67, 3.10, 5.55, 6.81, 12.5-13.0

¹³C-NMR (100 MHz, DMSOd₆) δ: 36.5, 78.5, 125.9, 157.9, 162.1, 170.4, 170.8

MS m/z: 330 (M⁺) (as TMS (trimethylsilyl) form of 3-carboxymuconolactone)

What is claimed is:

1. A protocatechuate 4,5-ring-cleaving enzyme gene-disrupted strain in which the gene coding for:
(a) the amino acid sequence set forth in SEQ ID NO: 1 or 3, or
(b) the amino acid sequence set forth in SEQ ID NO: 1 or 3 which has a deletion, substitution, addition and/or insertion of one or more amino acids and exhibits protocatechuate 4,5-ring cleavage activity,
present in the chromosomal DNA of microbial cells, has been disrupted.

2. A protocatechuate 4,5-ring-cleaving enzyme gene-disrupted strain in which a gene that:
(a) comprises the nucleotide sequence set forth in SEQ ID NO: 2 or 4; or
(b) is a nucleotide sequence hybridizing with DNA consisting of a nucleotide sequence complementary to the nucleotide sequence of (a), under stringent conditions, and coding for an enzyme with protocatechuate 4,5-ring cleavage activity,
present in the chromosomal DNA of microbial cells, has been disrupted.

3. A gene-disrupted strain according to claim 1 or 2, in which the protocatechuate 4,5-ring-cleaving enzyme gene has been disrupted by homologous recombination between a gene coding for:
(a) the amino acid sequence set forth in SEQ ID NO: 1 or 3, or
(b) the amino acid sequence set forth in SEQ ID NO: 1 or 3 which has a deletion, substitution, addition and/or insertion of one or more amino acids and exhibits protocatechuate 4,5-ring cleavage activity,
present in the chromosomal DNA of microbial cells, and homologous recombination DNA having a DNA sequence that can undergo homologous recombination with the gene and lacking protocatechuate 4,5-ring cleavage activity.

4. A gene-disrupted strain according to any one of claims 1 to 3, wherein the parent strain of the protocatechuate 4,5-ring-cleaving enzyme gene-disrupted strain is a Comamonas sp. bacterium.

5. A gene-disrupted strain according to claim 4, wherein the Comamonas sp. bacterium is Comamonas sp. E6.

6. A recombinant plasmid comprising a terephthalate dioxygenase gene (TPA-DOX gene), NADPH-reductase gene, 1,2-dihydroxy-3,5-cyclohexadiene-1,4-dicarboxylate dehydrogenase gene (DCD dehydrogenase gene), positive regulator gene, terephthalate transporter gene (TPA transporter gene) and protocatechuate 3,4-dioxygenase gene (pcaHG gene).

7. A transformant obtained by introducing a recombinant plasmid according to claim 6 into a gene-disrupted strain according to any one of claims 1 to 5.

8. A process for production of 3-carboxy-cis,cis-muconic acid and/or 3-carboxymuconolactone, characterized by culturing a transformant according to claim 7 in the presence of terephthalic acid.
